SEQUENCE LISTING (13 sequences)

SEQ ID NO: 1 - RNA, 24 nt, Homo sapiens
ugccugggucucuggccugcgcgu

SEQ ID NO: 2 - RNA, 19 nt, Homo sapiens
guccggucucuggguccgu

SEQ ID NO: 3 - RNA, 17 nt, Homo sapiens
cgggccaggcccaggcg

SEQ ID NO: 4 - RNA, 23 nt, Homo sapiens
ugcgcguccggucucuggguccg

SEQ ID NO: 5 - DNA, 22 bp, Homo sapiens
acaggctggcacagccccaggc

SEQ ID NO: 6 - RNA, 21 nt, Homo sapiens
cgcguccggucucuggguccg

SEQ ID NO: 7 - DNA, 21 bp, Homo sapiens
gtgcagcccagagacccaggc

SEQ ID NO: 8 - RNA, 20 nt, Homo sapiens
gcguccggucucuggguccg

SEQ ID NO: 9 - DNA, 20 bp, Homo sapiens
tgtctgccgggggcccaggc

SEQ ID NO: 10 - RNA, 20 nt, Homo sapiens
cguccggucucuggguccgu

SEQ ID NO: 11 - DNA, 21 bp, Homo sapiens
gcacactaggaggcccaggcg

SEQ ID NO: 12 - RNA, 23 nt, Homo sapiens
gcgcguccggucucuggguccgu

SEQ ID NO: 13 - DNA, 22 bp, Homo sapiens
cgctggggtggaggcccaggca

(19) United States
(12) Patent Application Publication
(10) Pub. No.: US 2012/0115928 A1
(43) Pub. Date: May 10, 2012

(54) MIRNA AND ITS TARGETS RESPECTIVELY THE PROTEINS MADE BASED ON THE TARGETS AS A PROGNOSTIC, DIAGNOSTIC BIOMARKER AND THERAPEUTIC AGENT FOR CANCER

(75) Inventors: Guillaume Vetter, Thionville (FR); Evelyne Friederich, Esch-sur-Alzette (LU)

(73) Assignee: UNIVERSITE DU LUXEMBOURG, Luxembourg-Limpertsberg (LU)

(21) Appl. No.: 13/260,488

(22) PCT Filed: Mar. 26, 2010
(86) PCT No.: PCT/EP2010/054041

§ 371 Date: Sep. 26, 2011

(30) Foreign Application Priority Data
Mar. 27, 2009 (LU) 91545

(51) Int. Cl. A61K 31/7088; A61P 25/00; G01N 33/574; A61P 35/00; C07H 21/02; C07K 14/47

(52) U.S. Cl. 514/44 A; 536/24.5; 530/350; 435/7.1; 435/7.23

(57) ABSTRACT

A compound miRNA (miRNA661) (Nr MI0003669 or ENSG00000207574) the sequence of the mature miRNA-661 being 51-ugccugggucucuggccugcgcgu-74 for use as a medicament in the in the treatment and/or the diagnosis of cancer, neuronal disease and infection.

### TECHNICAL FIELD

The present invention generally relates to a novel miRNA, the use of this novel miRNA and its targets respectively the proteins made based on the targets as a specific biomarker of cancer and in particular of adenocarcinoma breast cancer and the use of the novel miRNA to reduce the invasive and migratory potential of cancer cells.

### BACKGROUND

MicroRNAs (miRNAs) are ˜22nt-long noncoding RNAs that coordinate gene expression at the post-transcriptional level. These small RNAs are thought to inhibit virtually all steps of translation, from initiation to elongation, through imperfect micro-homologies with the 3′UTR (3′-untranslated regions) of the targeted messengers RNAs (mRNA). MiRNAs can also elicit the destabilization following by the degradation of mRNAs and the discovery of this later mode of action has greatly facilitated the understanding of their functions by appropriate large-scale techniques such as microarrays. In fact, around 700 miRNAs are known to exist in mammalian cells, each one having multiple targets and each mRNA being targeted by several miRNAs. This crucial contribution to fundamental cell functions implies that aberrant expression of miRNAs is often associated with pathologies, in particular cancers, or neuronal disease or infection. Indeed, a strong link between miRNA and human cancers is now well established, as miRNAs have been demonstrated to act as either oncogenes (also termed Oncomirs) (e.g., miR-155, miR-17-5p) or tumor suppressors (e.g., let-7, and miR-143/145). They also represent promising diagnostic and prognostic markers as well as novel targets of alternative therapeutic strategies.

It has been found that miRNAs are implicated in the tumoral progression of epithelial cancer cells also called Epithelial-to-Mesenchymal-Transition (EMT) (reviewed in (Cano & Nieto, 2008).

For example, signals triggering EMT lead to the down-regulation of the miR-200 family and miR-205, which is required for the maintenance of the epithelial phenotype.

Likewise, the miR-10b has been shown to trigger in vivo tumor invasion and metastasis of epithelial breast cancer cells.

Signals triggering EMT elicit the expression of transcription regulators such as SNAI1 that orchestrate key events of this process. SNAI1 induces EMT by directly binding to the promoter of epithelial genes to repress their transcription. In addition, ectopic expression of SNAI1 is known to confer invasive behavior to cell lines from various origins. On the other hand, silencing of SNAI1 in highly invasive MDA-231 human breast cancer cell line markedly diminished cell invasion in vivo and in vitro.

WO20080144047 is related to compositions and methods for delivering an agent to a cell comprising a prolactin receptor. It is a method of inhibiting a breast, ovarian or prostate cancer cell, where the method includes a step of contacting the cell with a complex comprising a prolactin receptor ligand linked to at least one of an RNAi-inducing agent. The RNAi-inducing agent being a polynucleotide sequence encoding a polypeptide, an miRNA, a cytotoxic moiety, a chemotherapeutic moiety, a radioactive moiety or a nanoparticle. Methods of detecting a cancer cell expressing a prolactin receptor are also disclosed. However this method does not cite using a specific miRNA for the detection of breast cancer.

WO2008137867 relates to compositions comprising miR-34 and siRNAs functionally and structurally related to miR-34 for the treatment of cancer—

US20060078906 discloses a specific method for detecting target polynucleotide such as mi-RNA that can target mRNAs for cleavage and attenuate translation.

It should be pointed out that some miRNAs have been described as related to human cancer, namely blood cancer such as leukemia (ALL and B-CLL), T cell leukemia, APL (AML3), CML or tumors such as malignant lymphoma, Burkitt lymphoma, breast cancer, cholangiocarcinoma, colorectal cancer, follicular thyroid carcinoma, hepatocellular carcinoma, neuroblastoma, non-small cell lung cancer, ovarian cancer, pancreatic cancer, papillar thyroid carcinomas, pituitary adenomas, prostate cancer, stomach cancer, testicular germ cell tumours, thyroid anaplastic carcinomas, (reviewed in Saumet et al., 2008, Table 1). However, few are known to be specifically associated to breast cancer; among them miR-10b, miR-17-5p, miR-125b, miR-143, miR-145 are reported to be downregulated and are tumor suppressors, whereas miR-21 miR-29b miR-146 miR-155BIC are known to be up-regulated and are oncogene.

Consequently, it is an object of the present invention to propose a novel miRNA related to cancer (diagnosis and cure) and more specifically to epithelium derived carcinomas.

### BRIEF SUMMARY OF THE INVENTION

In order to overcome the above-mentioned problem, the present invention proposes miRNA661 according to SEQ ID Nr MI0003669 (miRbase; http://microrna.sanger.ac.u) or ENSG00000207574 (http://www.ensembl.org) 51-ugccugggucucuggccugcgcgu-74 for use as a medicament.

The present invention concerns miRNAs, this miRNA sequence was found by large-scale experimental cloning of novel human miRNAs in human colorectal tissue (Cummins et al, 2006). However, this miR661 have no assigned functions yet. It has now been found that it participates in epithelial to mesenchymal transition of cancer cells and more specifically to epithelium derived carcinomas, the epithelial to mesenchymal transition being a key step of carcinoma cell progression towards an invasive state. In particular, miR661 participates in epithelial to mesenchymal transition of breast cancer cells. Furthermore, this miR661 is expressed in colorectal cancer.

The present invention is related to a specific prognosis and diagnosis use of miRNA661 and its targets respectively the proteins made based on the targets in particular for invasion and migration of cancer and more particularly to metastasis carcinoma breast cancer.

It has been found that invasive breast cancer carcinoma cell lines are associated with overexpression of miRNA 661 and down regulations of its targets: Nectin-1 (or PVRL1) (refSequence: NM_{—}002855; ensembl: ENST00000264025) and StarD10 (refSequence: NM_{—}006645; ensembl: ENST00000334805) mRNAs and their corresponding proteins down expressions.

Inhibition with classic and known methods of miRNA associated EMT, such as miRNA 661 inhibits invasion of breast cancer and is used as therapy strategy for inhibiting metastasis correlated with death patient.

Hence, it is one aspect the present invention to use miR-661 and its associated targets (Nectin-1 and StarD10) respectively the proteins made based on the targets as a breast cancer prognostic and diagnostic tool.

In another aspect, the present invention is related to a specific biomarker of invasive breast cancer cells whose expression positively participates in elicitating cell migration and invasion.

In another aspect the present invention is related to a therapeutic method for treating human pathologies, which comprises the blocking of miRNAs function (in vivo and in vitro) with LNAs or other compounds in cancer, neuronal disease or infection to inhibit the synthesis of miRNAs corresponding targets, namely mRNAs and their corresponding proteins transcripts (see miRNA associated therapies).

In another aspect the present invention is related to a therapeutic method for treating adenoma breast cancer which comprises the blocking of miRNA 661 (In vivo and in vitro) with LNAs in invasive breast cancer carcinoma cell lines to inhibit the synthesis of its targets adherens junction proteins Nectin 1 and StarD10, by hybridization and silencing of Nectin 1 and StarD10 mRNAs and their corresponding transcripts (see mi RNA associated therapies).

In another aspect the present invention provides a method, kits and devices for identifying biomarker miRNAs of treatment response and miRNAs expression variation associated with human pathologies such as cancer, neuronal disease and infection.

In another aspect the present invention provides/features a method, kits and devices for identifying biomarker miRNA-661 of treatment response and miRNA-661 expression variation associated with human pathologies such as cancer, and specifically adenocarcinoma breast cancer.

In another aspect the present invention provides/features a method, kits and devices for identifying and quantifying biomarker miRNA-661, its associated targets (Nectin-1 and StarD10) respectively the proteins made based on the targets.

In yet another aspect a cell based model (MCF-7-SNAI 1 recapitulating EMT) of an invasive breast adecarcinoma cancer model has been established to clarify the mechanism of contribution of miRNAs and miRNA 661 to the initiating events of EMT and cell invasion and to develop a therapeutic agent for adenoma breast cancer treatment cancerated by SNAI 1. Further the invasive breast cancer cell line produces an overexpression of miRNA 661 whose quantification is indicative of invasion and migration and therefore metastasis in tumoral progression of adecarcinoma breast cancer.

In another aspect the present invention provides a novel miRNA quantification method based on a specific Reverse Transcriptase (RT) followed by Polymerase Chain Reaction (PCR) amplification (FIG. 1). Stem-loop RT primers are designed to bind to the 3′ region of miRNA molecules which are reverse transcribed with regular reverse transcriptase. The stem-loop RT primers are better than conventional ones in terms of RT efficiency and specificity. The RT product is quantified using conventional quantitative PCR using miRNA-specific forward primer and a reverse primer complementary to the stem-loop oligonucleotide used for the RT. These miRNA assays are specific for mature miRNAs and can discriminate related miRNAs that differ by one nucleotide. These assays are not affected by genomic DNA contamination. Precise quantification is achieved routinely with as little as 250 ng of total RNA for most miRNAs. This method enables fast, accurate and sensitive miRNA expression profiling and can identify and monitor potential biomarkers specific to tissues or diseases.

### BRIEF DESCRIPTION OF THE FIGURES

Further details and advantages of the present invention will be apparent from the following detailed description of several not limiting embodiments with reference to the attached figures, wherein:

FIG. 1 shows the Correlation of SNAI1-expression kinetics with phenotypic changes and gain of invasive capacity in MCF-7-SNAI cells, in particular:

FIG. 1A shows PCR increased expression of SNA I1 in inducible MCF-7SNA11 cell lines with a half maximum value after 8 hours after induction.

FIG. 1B shows DAPI staining and immunofluorescence decrease of epithelial proteins E cadherin and Cytokeratin 18 associated with change of epithelium to mesenchymal phenotype 24 hours after SNAI1 induction.

FIG. 1C shows light microscopy change of epithelium to mesenchymal phenotype 24 hours after SNAI1 induction,

FIG. 1D shows DAPI staining and immunofluorescence expression of SNAI1 concentration in the nucleus.

FIG. 1E—shows Texas red phalloidin staining apparition of stress fibers and reorganisation of the actin cytoskeleton in the induced MCF-7SNAI1 cells.

FIG. 1F shows Transwells assays increase of the MCF-7-SNAI1 cell migration and cell invasion into Matrigel, 48 H post-induction.

FIG. 2—miR-661 early up-regulation after SNAI-induction and over expression in invasive breast cancer cells is necessary but not sufficient for the cell migration and invasion, in particular:

FIG. 2A shows miRNA-microarrays results obtained 8 hours after SNAI1 induction in MCF-7-SNAI1 and shows down regulation of following miRNAs: miR-141, miR-200c, miR-200a, miR-200b and miR-429 and miR-205 and up regulations of following miRNAs: miR-424, miR-661 and miR-940.

FIG. 2B shows Realtime RT-PCR increase of miR-940, miR-424 and miR-661 expression 8 hours after SNAI1 induction.

FIG. 2C shows Real time RT-PCR increase of miR-661 expression in invasive cells versus non-invasive cells.

FIG. 2D shows Realtime RT-PCR monitoring of miR-661 expression in MCF-7-SNAI1 cells and its early up-regulation (4 h after induction) by SNAI1 in triggering EMT.

FIGS. 2E and 2F show Transwell migration assay and Matrigel invasion assay of MCF-7-SNAI1 induced (E) and MDA-435 cells (F) transfected by the specific LNA antisens of miR-661 (LNA-661), or with the scrambled LNA (LNA-sc) as a control

FIG. 2G shows Transwell migration assay and Matrigel invasion assay of MCF-7 transfected with pSuper-miR-661 or empty vector (pSuper-empty) and shows no significantly modification of their phenotype.

FIG. 2H shows Real-time PCR of miR-661 in MCF-7 transfected 24 h or 48 h by pSuper-miR-661 and shows forced expression of miR-661 (coined pSuper-miR-661 vector) in the weakly invasive MCF-7 breast cancer epithelial cell line without significantly modification of their phenotype or migratory or invasive behaviour.

FIG. 3 show that miR-661 regulates negatively the Nectin-1 and StarD10 expression during SNAI1 induced-EMT of MCF-7 cells, in particular:

FIG. 3A shows Predicted In Silico mRNA 3′UTR targets binding sites of miR-661 realized using miRBase Target Version 5 (http://micrornasanger.ac.uky): 28 genes have been identified comprising StarD10 FLII, Nectin-1, RNPEL1, NQ2, CACNAH1H.

FIG. 3B shows evaluation of the messenger RNA level by Realtime PCR (up) and protein level by immunoblot (down), 48 h after forced expression with pSuper-miR-661 vector or pSuper-empty as a control in MCF-7 cells and shows down regulation of Nectin-1 and StarD10 mRNAs and proteins.

FIG. 3C shows phalloidin staining and immunoflorescence with anti-SNAI1 antibody and with anti-nectin-1 (up) or anti-StarD10 (down) and reveals the expression of SNAI1 48 H after the removal of Tetracycline (induced) and the decrease of the Nectin-1 and StarD10 expression, compared to the non-induced cells in presence of Tetracyclin.

FIG. 3D shows WesternBlots decrease of Nectin-1 and StarD10 proteins expressions after induction (−tet) in MCF7-SNAI1 expressing miR-661 versus no decrease of Nectin-1 and StarD10 proteins expressions in inducted MCF7-SNAI-1 where expression of miR-661 is inhibited by antisense LNA-661.

FIG. 3E shows inhibition of miRNA661 by antisense LNA-661 in induced MCF7-SNAI1 and shows quantification of Nectin-1 and StarD10 RNAs. Real-time PCR assays realized on mir661 candidates targets genes mRNAs (i.e., NOQ2, StarD10, FLII, RNPEPL1, Nectin-1, and CACNAH1) or E-cadherin as a control after transfection of LNA-661 or LNA-sc as a control in induced MCF-7-SNAI1 (−Tet) or non-induced MCF7-SNAI1 (+Tet) cells and shows a decrease of Nectin-1 and StarD10 mRNAs and their destabilization in MCF-7-SNAI-1 induced cells treated with the control LNA sc (LNA scrambled) expressing miRNA 661 and a protection of the said RNAs targets from destabilization when protected by LNA-661 (or anti miRNA-661 LNA).

FIG. 4. Nectin-1 and StarD10 are down-regulated early after SNAI1 induction in MCF-7-SNAI1 cells and are expressed in normal or cancer epithelial cells but not in fibroblastic-like breast cancer cells, in particular:

FIG. 4A. Detection of Nectin-1 and StarD10 mRNAs by Real-time PCR in breast cancer cell lines of varying invasive character: non-invasive cells such as HMEC or MCF10F or weakly invasive cells such as T47D or MCF-7 and highly invasive cells such as MDA-435 and MDA-231

FIG. 4B. Detection of Nectin-1 and StarD10 mRNAs by Real-time PCR in MCF7-SNAI1 cells show that Nectin-1 mRNA level decreased between 8 h and 12 hours after SNAI1 expression and StarD10 level between 12 h and 24 h in induced MCF-7-SNAI1 cells, and suggesting an early regulation.

FIG. 4A and FIG. 2C show inverse correlation between the expression of miR-661 and the expression of Nectin-1 and StarD10 in non-invasive and invasive epithelial cell lines.

FIG. 5 shows the expression of epithelial and mesenchymal markers after SNAI1 induction in MCF7-SNAI1 cells and the expression of SNAI1 in breast cancer cell lines, in particular:

FIG. 5A. Detection by Real-time PCR of mRNA expression in induced MCF7-SNAI1 cells from compounds of adherens junctions (E-cadherin), tight junctions (Claudin-3, ZO-1), desmosomes (desmoplakin) and intermediates filaments (cytokeratin-18, cytokeratin-8), and the expression of mesenchymal markers such as N-cadherin, mmp-2, Zeb1, and SNAI2.

FIG. 5B Detection of the protein expression of SNAI1, E-cadherin and cytokeratin-18 (KRT18) in non-induced MCF7-SNAI1 (+tet) and induced MCF7-SNAI1 (−tet) cells

FIG. 5C Detection of SNAI1 mRNA by Real-time PCR in breast cancer cell lines of varying invasive character: non-invasive cells such as HMEC or MCF10F or weakly invasive cells such as T47D or MCF-7 and highly invasive cells such as MDA-435 and MDA-231.

FIG. 6: Evaluation of StarD10 expression in human breast tumors Expression of StarD10 in basal-like (BL), Luminal A, B (LA and LB), normal-like breast (NBL) and Her2+ (HR) breast tumors subtypes of 295 human breast tumors characterized in a previous study (van de Vijver et al., 2002).

Table 2 shows candidate target genes, which were also found to be down regulated in DNA-microarrays.

### DETAILED DESCRIPTION OF THE INVENTION AND EXAMPLES

The following examples are provided to assist in a further understanding of the invention. The particular materials and conditions employed are intended to be further illustrative of the invention and are not limiting on the reasonable scope of the appended claims.

Definition of Terminology

As used herein, the term “Neuronal disease” refers to diseases of nervous system's development such as mental deficiency, autism, schizophrenia and neurodegenerative diseases, such as Alzheimer's and Parkinson's diseases and any Neuronal disease associated with miRNAs variation of expression.

As used herein the “Metabolism” refers to lipid metabolism and diabetes and any metabolism associated with miRNAs variation of expression.

As used herein the term “infection” refers to immunity and viral infections, wherein the term immunity comprises cells of the immune system such as B and T lymphocytes and dendritic cells.

As used herein, the term “microRNA species”, “microRNA”, “miRNA”, or “mi-R” refers to small, non-protein coding RNA molecules that are expressed in a diverse array of eukaryotes, including mammals. MicroRNA molecules typically have a length in the range of from 15 to 120 nucleotides, the size depending upon the specific microRNA species and the degree of intracellular processing. Mature, fully processed miRNAs are about 15 to 30, 15 to 25, or 20 to 30 nucleotides in length, and more often between about 16 to 24, 17 to 23, 18 to 22, 19 to 21, or 21 to 24 nucleotides in length. MicroRNAs include processed sequences as well as corresponding long primary transcripts (pri-miRNAs) and processed precursors (pre-miRNAs). Some micro RNA molecules function in living cells to regulate gene expression via RNA interference. A representative set of microRNA species is described in the publicly available miRBase sequence database as described in Griffith-Jones et al., Nucleic Acids Research 32:D109-D111 (2004) and Griffith-Jones et al., Nucleic Acids Research J4:D140-D144 (2006), accessible on the World Wide Web at the Wellcome Trust Sanger Institute website.

As used herein, the term “locked nucleic acid” (LNA), often referred to as inaccessible RNA, is a modified RNA nucleotide. The ribose moiety of an LNA nucleotide is modified with an extra bridge connecting the 2′ and 4′ carbons.

As used herein, the term “RNA blocking” or “RNAi/LNA” refers to the silencing or decreasing of gene expression by iRNA/LNA agents (e.g., siRNAs, miRNAs, shRNAs), via the process of sequence-specific, post-transcriptional gene silencing in animals and plants, initiated by an iRNA/LNA agent that has a seed region sequence in the iRNA/LNA guide strand that is complementary to a sequence of the silenced gene. LNA oligonucleotide used to inhibit miRNA function possesses the exact antisense sequence of the corresponding mature miRNA. The specific antisens LNA binds to the mature miRNA and hindered its silencing function (i.e. inhibition or activation of translation, mRNA destabilization, induction of encoding-gene expression)

As used herein, the term an “iNA agent” (abbreviation for “interfering nucleic acid agent”), refers to an nucleic acid agent, for example RNA, or chemically modified RNA, which can down-regulate the expression of a target gene. While not wishing to be bound by theory, an iNA agent may act by one or more of a number of mechanisms, including post-transcriptional cleavage of a target mRNA, or pre-transcriptional or pre-translational mechanisms. An iNA agent can include a single strand (ss) or can include more than one strands, e.g., it can be a double stranded (ds) iNA agent.

As used herein, the term “single strand iRNA agent” or “ssRNA” is an iRNA agent which consists of a single molecule. It may include a duplexed region, formed by intra-strand pairing, e.g., it may be, or include, a hairpin or panhandle structure. The ssRNA agents of the present invention include transcripts that adopt stem-loop structures, such as shRNA or Pre-miRNA., that are processed into a double stranded siRNA or single stranded miRNA respectively,

As used herein, the term “ds iNA agent” is a dsNA (double stranded nucleic acid (NA)) agent that includes two strands that are not covalently linked, in which interchain hybridization can form a region of duplex structure. The dsNA agents of the present invention include silencing dsNA molecules that are sufficiently short that they do not trigger the interferon response in mammalian cells.

Results

Correlation of SNAI1-expression kinetics with phenotypic changes and gain of invasive capacity in MCF-7-SNA1 cells

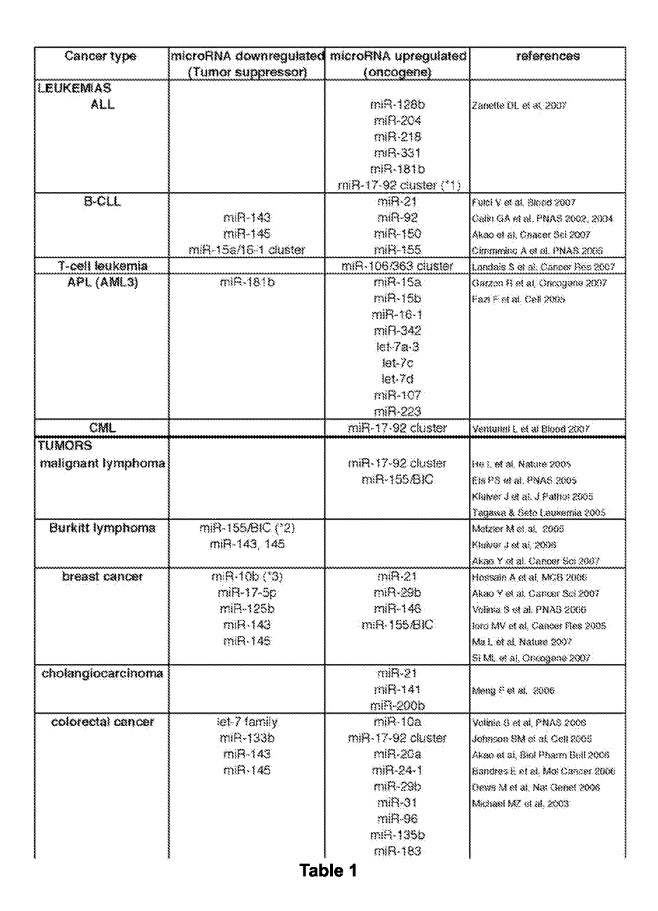

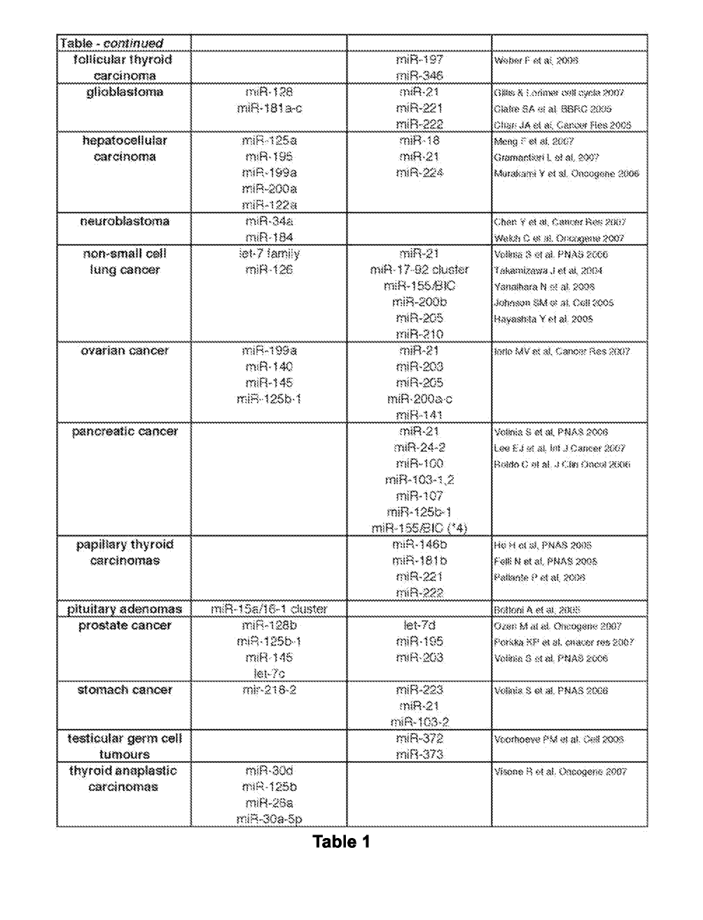

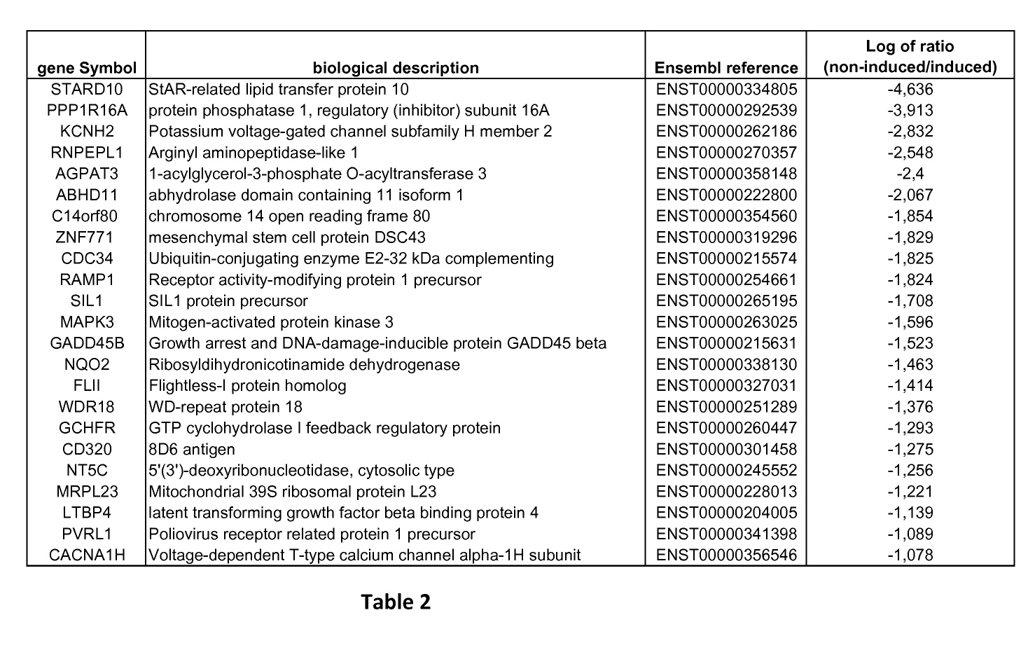

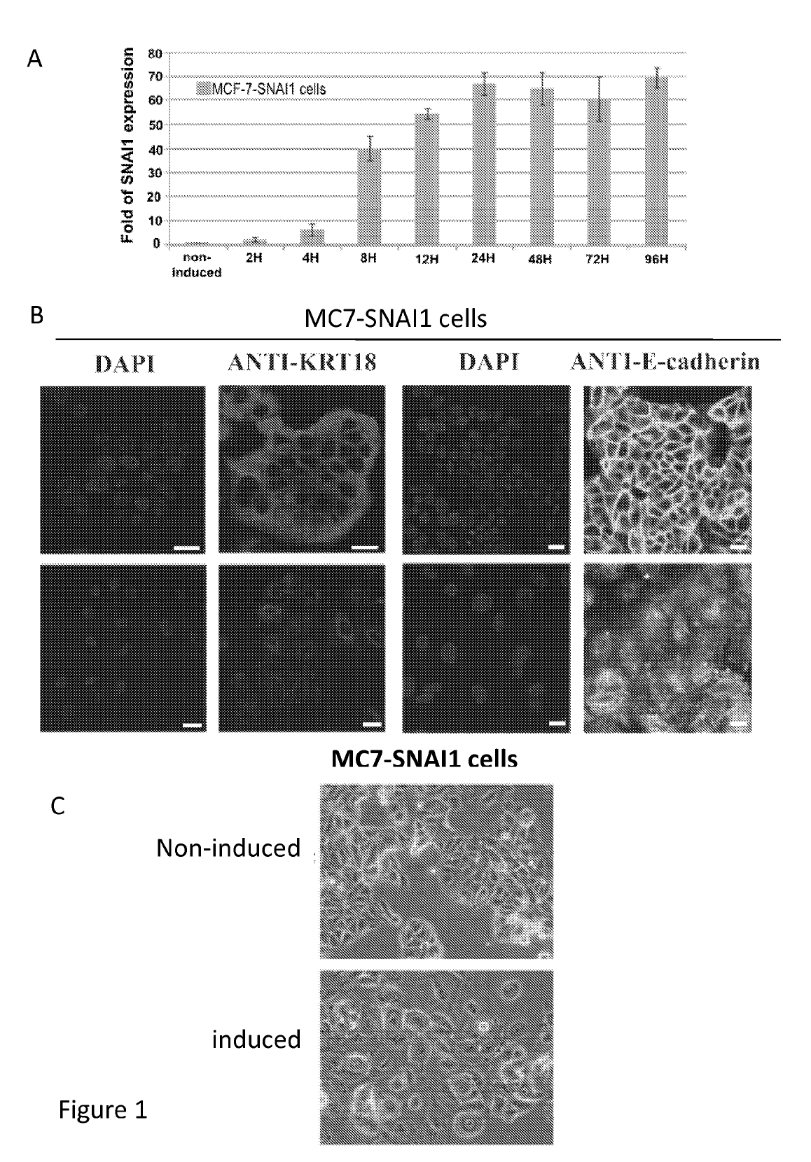

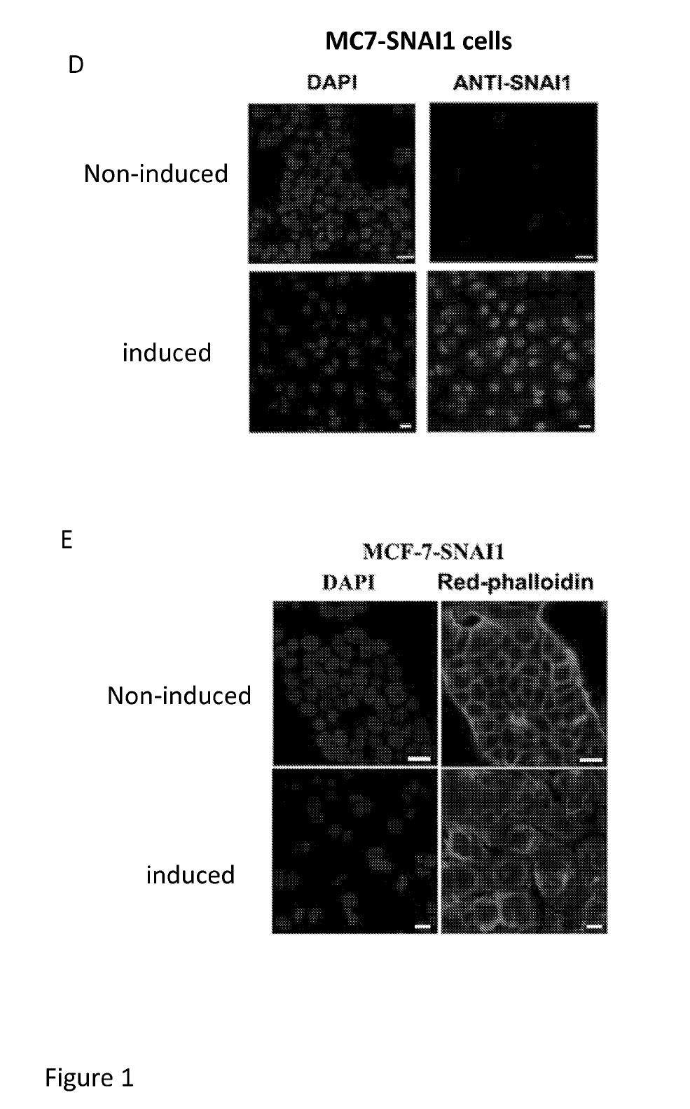

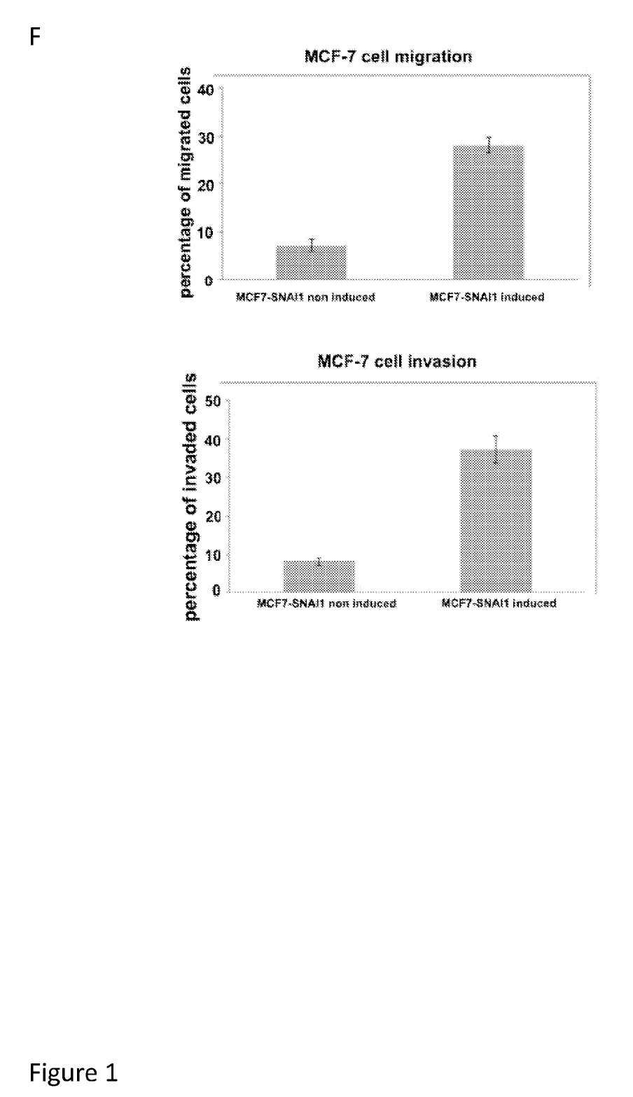

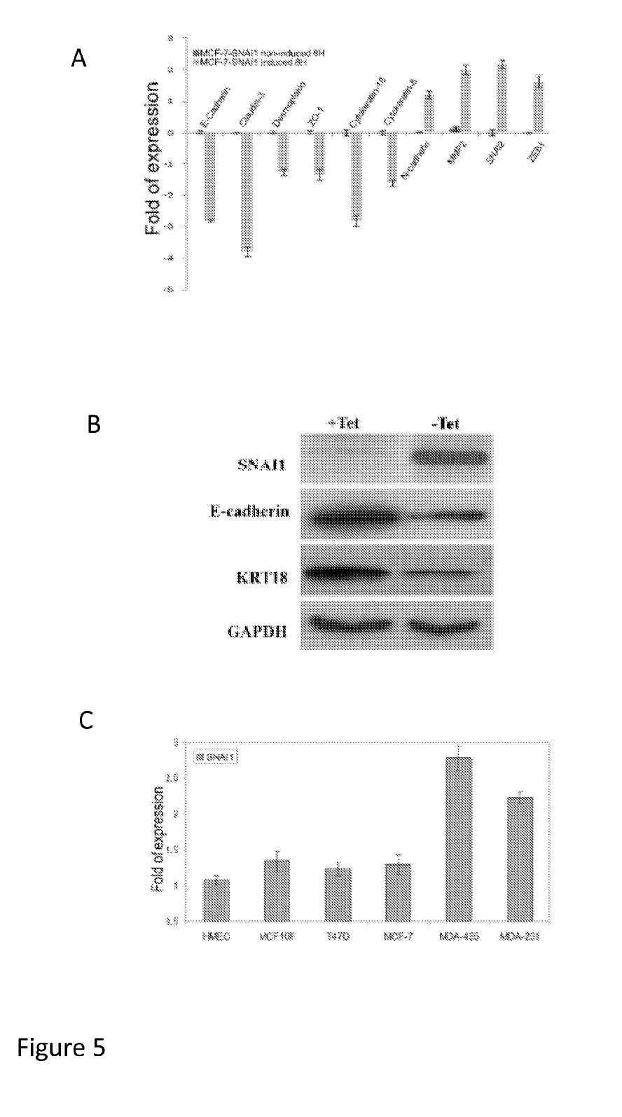

To identify miRNAs which are differentially expressed during EMT, MCF-7-tetoff cells conditionally expressing human full-length SNAI1 cDNA under the control of a responsive tetracycline operator element have been used. These cells which were called MCF-7-SNAI1 were used in time course experiments to study early events accompanying EMT. SNAI1 expression was detected 2 hours after the removal of tetracycline from the medium (−Tet, and referred here to as induction) and its level increased as a function of time, with a half-maximal value at 8 hours (FIG. 1A). Changes in the expression of specific epithelial and mesenchymal marker genes could be observed at early time points, 8 hours after induction (data not shown). The induction of SNAI1 led to global decrease in the expression of epithelial genes 48 Hours post-induction, i.e compounds of adherens junctions (E-cadherin), tight junctions (Claudin-3, ZO-1), desmosomes (desmoplakin) and intermediates filaments (cytokeratin-18, cytokeratin-8). Conversely, the expression of mesenchymal markers such as N-cadherin, mmp-2, Zeb1, and SNAI2 was up-regulated (FIG. 5A). The repression of E-cadherin and cytokeratin-18 were also confirmed, at the protein level (FIG. 5B). Typical changes in cell morphology were observed 24 hours after induction: non-induced cells form characteristic epithelial cell clusters whereas induced MCF7-SNAI1 cells exhibit fibroblastic-like scattered colonies (FIG. 1C). These changes became more pronounced after prolonged SNAI1 expression. Expression of SNAI1 which is concentrated in the nucleus (FIG. 1D) induced the loss of cell-cell contacts and a switch to a mesenchymal cell phenotype as also evidenced by the reorganisation of the actin cytoskeleton and the appearance of stress fibers (FIG. 1E). As expected, SNAI1 expression highly increased the capacity of non-transformed MCF-7 cells to invade Matrigel and to migrate in a transwell assay when compared with non-induced cells (FIG. 1F). Together, the observations made demonstrate that inducible expression of SNAI1 in MCF-7 cells allows studying the initiating events of EMT and cancer cell invasion and therefore constitutes a valuable model to identify miRNAs involved therein.

The miR-661 is up-regulated at early time points after SNAI-induction and is highly expressed in invasive breast cancer cells

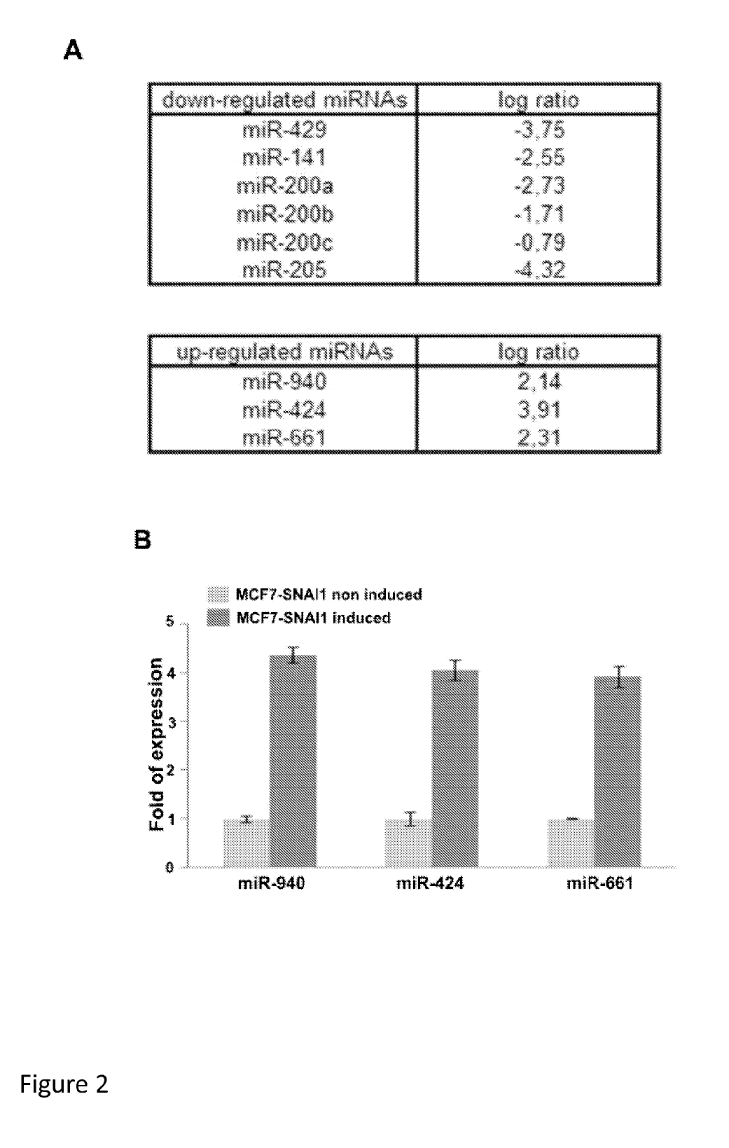

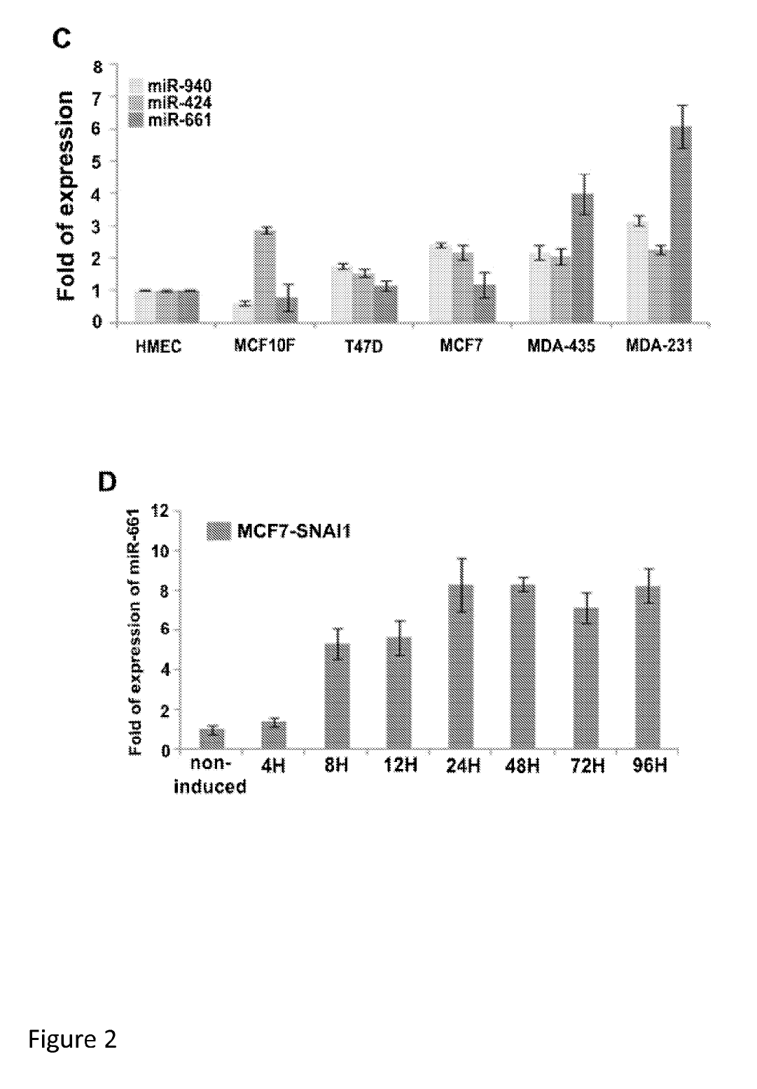

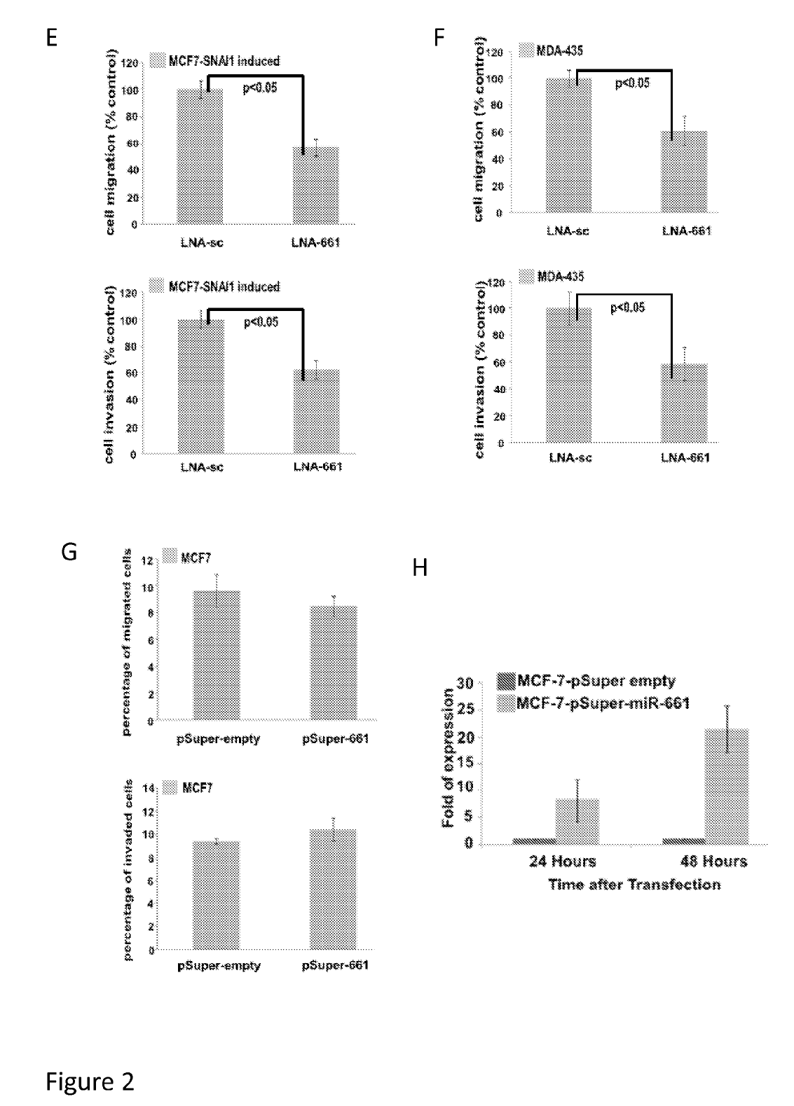

To identify miRNAs the expression of which are modulated during the initiating events of SNAI1 -induced EMT, microarray-based miRNA profiling at 8 hours post-induction was performed, a time point where changes in the expression profiles of mRNA-encoding genes are observed. It was observed that 72 human mature miRNAs out of 453 were differentially expressed. Interestingly, at this early time point, the expression of the miR-141, miR-200c, miR-200a, miR-200b and miR-429 and miR-205 were found down-regulated (FIG. 2A). These findings were consistent with previous reports showing that the repression of these six miRNAs is required for epithelial differentiation (reviewed in (Cano & Nieto, 2008) and documented in Gregory et al., 2008; Korpal et al., 2008). In order to identify new miRNAs that positively contribute to epithelial cell dedifferentiation and cell invasion, it was decided to focus on up-regulated miRNAs that were not reported until now i.e. miR-424, miR-661 and miR-940. Using quantitative stem-loop polymerase chain reaction (RT-qPCR), it was verified that SNAI1 induction readily induced the up-regulation of the miR-424, miR-661 and miR-940 8 Hours after induction (FIG. 2B). To examine whether the up-regulation of these 3 miRNAs correlated with the invasive behavior of breast cancer cells, their expression in non-invasive normal immortalized breast cancer cell lines (HMEC (derived from patient), MCF10F (ATCC CRL-10318)), weakly (T47D (ECACC 85102201), MCF-7 (ECACC 86012803) and highly invasive cell lines (MDA-435 (ATCC number CRL-2914) MDA-231(ATCC number HTB-26,)) which exhibited high levels of SNAI1 expression ((Bathe et al, 2000))(FIG. 2C and FIG. 5C) was analyzed. Among these three miRNAs, only miR-661 was found highly expressed in invasive cells compared to non invasive HMECs (FIG. 2C).

Since miR-661 exhibited an expression pattern closely linked to the degree of invasion, it was decided to characterize more in detail this miRNA. Monitoring the expression of miR-661 during SNAI1-triggered EMT revealed that its up-regulation started 4 hours after induction, increased throughout time and remained at a high level at later time points (96 hours) suggesting that its early and sustained expression is required for SNAI1 induced EMT (FIG. 2D). This result and the observation that expression of miR-661 closely correlated with the invasive capacity of breast cancer cells, prompted to further investigating its contribution to breast cancer cell invasion trigged by SNAI1 expression.

Inhibition of the miR-661 decreases the migration and invasion properties of invasive breast cancer cells

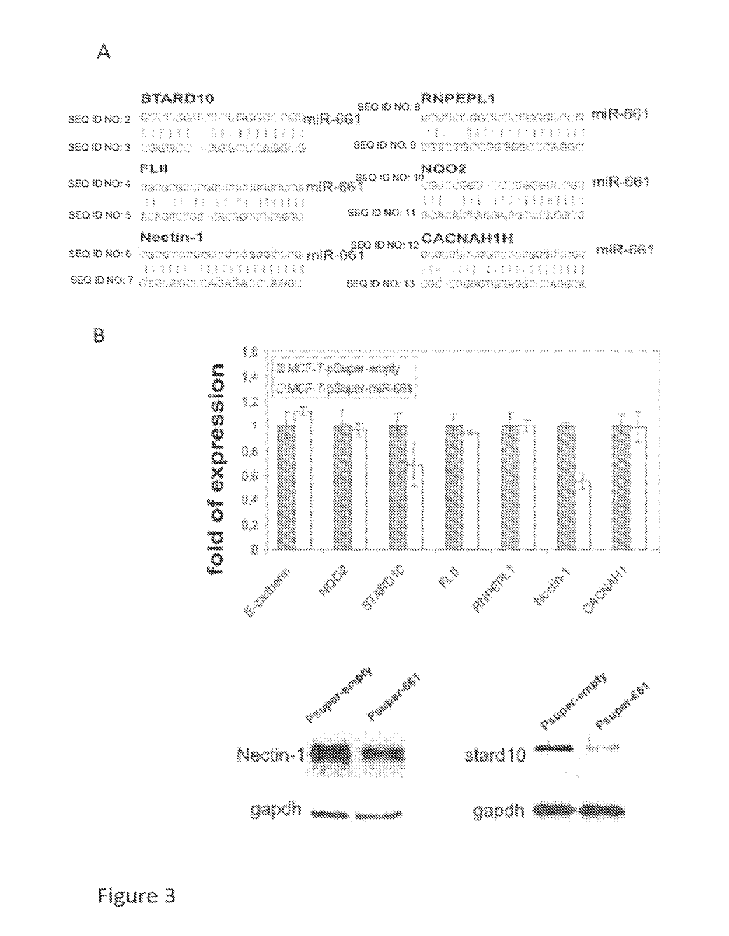

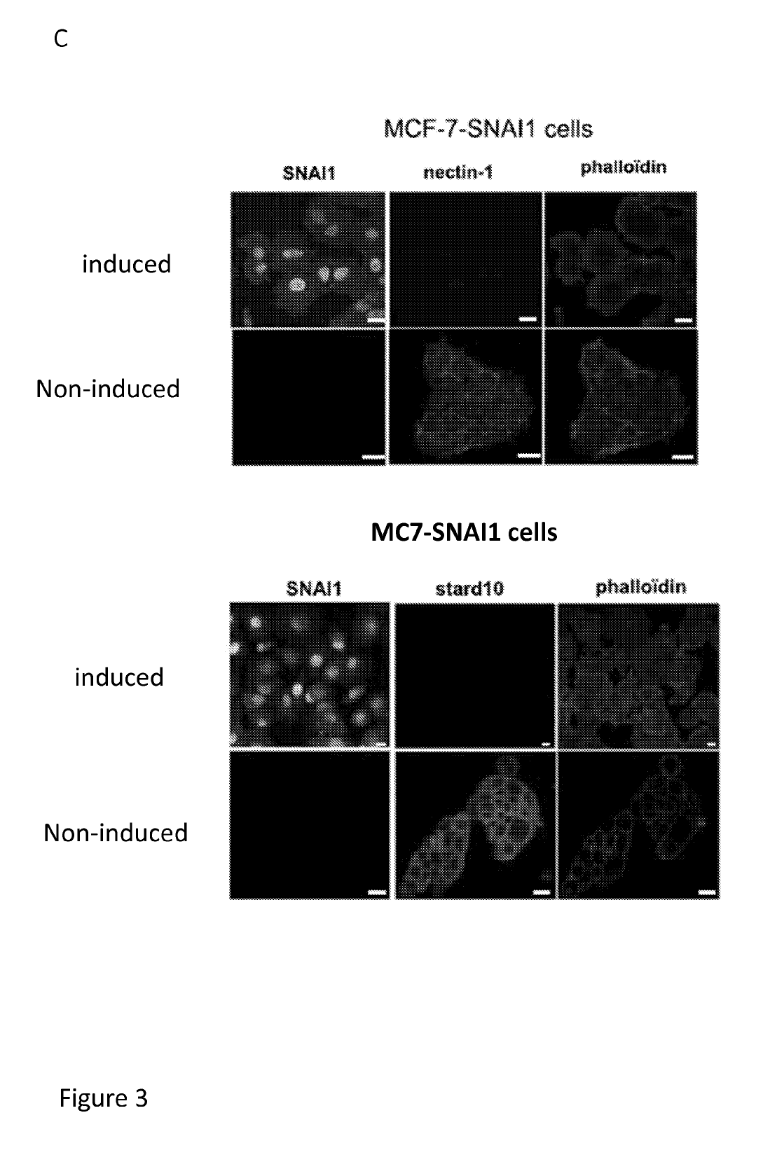

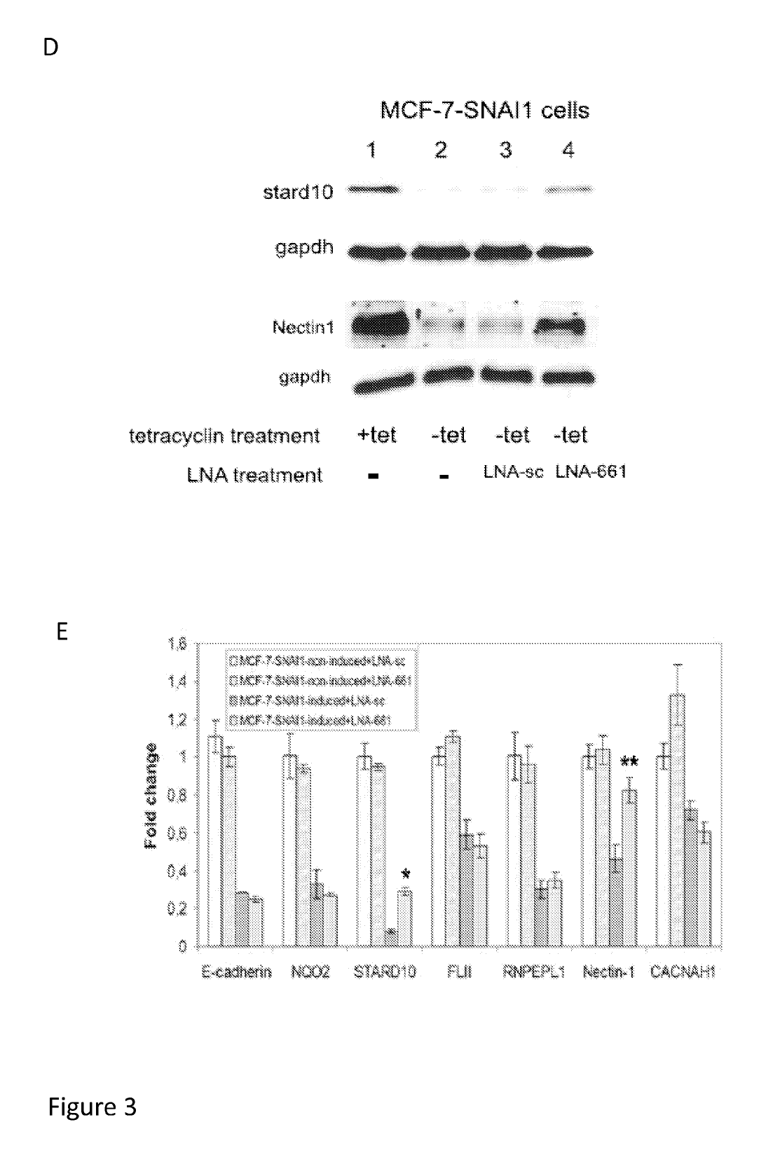

To test whether miR-661 was involved in EMT-associated breast cancer cell invasion, its action was inhibited by transfecting MCF-7-SNAI1 cells with specific antisense Locked Nucleic Acids (LNA) oligonucleotides of the miR-661 (LNA-661) prior to SNAI1 induction or in the invasive MDA-435 cells expressing SNAI1 and the miR-661. The use of Cy3-coupled LNAs allowed determining the efficiency of transfection of MCF-7-SNAI1 and MDA-435 cells, which reached 70% of total cells. Transfection with an LNA directed against the miR-661 (LNA-661) did not affect the cell phenotype, but reduced the cell migration and invasion of induced MCF-7-SNA11 by 40% and 35%, respectively, when compared to induced cells transfected with a scrambled LNA (LNA-sc), used as a control (FIG. 2E). Transfection with LNA-661 of highly invasive MDA-435 cells had no effect on cell phenotype but also reduced migration and invasion by 37% and 42%, respectively compared to the cells transfected by LNA-sc (FIG. 2F). It was verified that these effects were not due to a decrease of cell proliferation. Forced expression of miR-661 (coined pSuper-miR-661 vector) in the weakly invasive MCF-7 breast cancer epithelial cell line did not significantly modify their phenotype, migratory or invasive behavior (FIG. 3G). Expression of the miR-661 was monitored by real-time PCR (FIG. 2H).

Taken together, these observations suggested that although additional factors might be required to trigger breast cancer cell invasion, miR-661 expression is necessary for this process (FIG. 2E and FIG. 2F).

The miR-661 regulates negatively the Nectin-1 and StarD10 expression during SNAI1 induced-EMT of MCF-7 cells.

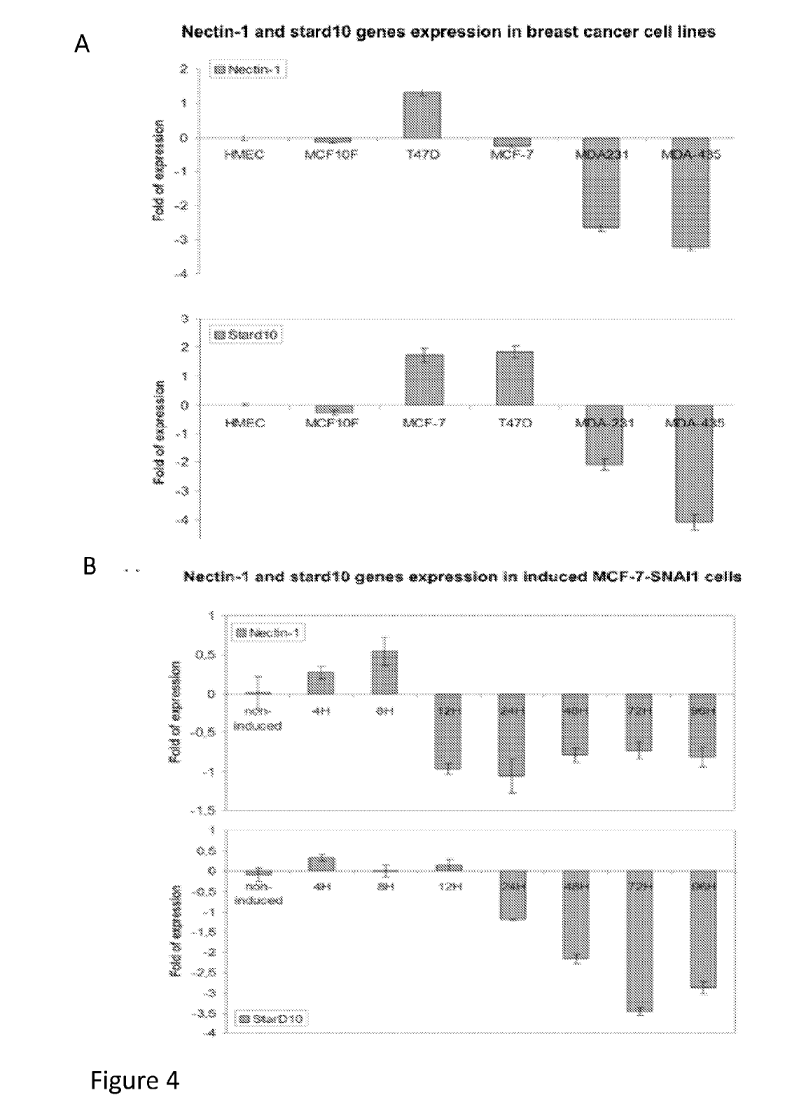

To further investigate the contribution of the miRNA-661 in the epithelial cell transition, it was decided to identify the corresponding mRNA targets, which are specifically negatively regulated by this miRNA. Because the matching between the miRNA sequence and its corresponding targets messengers sequences is not perfect, around 1000 of potential candidate target genes were predicted by in silico searches to be potentially regulated by the miRNA-661. To reduce the number of potential miRNA-661 targets, it was decided combining in silico approaches with transcriptomic analyses. Hence, since it is known that miRNAs repress protein expression either by blocking translation without affecting its targeted messenger level or by the destabilization of the targeted messenger RNA, the identification of candidate messengers with the latest assumption was started. It was reasoned that the up-regulation of miRNA-661 induced by SNAI1 should be accompanied by the destabilization of at least part of the targeted messengers. Such approach has already been shown to be an appropriate strategy to identify functionally important miRNA targets (Saumet et al, 2009)). Pan-genomic microarrays data obtained with MCF7-SNA1 cells revealed that 700 genes were down-regulated upon SNAI1 induction in MCF7-SNAI1 cells and could be potentially regulated by miR-661. Among those, 30 genes predicted to be targeted by miR-661 using the miRBase Targets Version 5, (http://microrna.sanger.ac.uk) (Table 2 and FIG. 3A) were selected. To evaluate whether miR-661 was, at least in part, responsible for the modulations of the levels of these candidate mRNAs, MCF7 cells were transfected with the pSuper-miR-661 or the pSuper-empty vectors and quantified the mRNA of these potential targets by RT-qPCR. As a negative control, the E-cadherin mRNA was used, which is directly down-regulated by SNAI1 ((Bathe et al, 2000)) but is not present in the list of the predicted targets of miRNA-661. Forced expression of miRNA-661 did neither change the level of E-cadherin mRNA, nor that of the 28 potential targets i.e. NQO2, FHLII and RNPL1 (FIG. 3B). Conversely to the other potential targets, it was found that Nectin-1 and StarD10 messengers RNA and protein levels decreased 48 hours after pSuper-miR661 transfection (FIG. 3B). This result suggested that these two genes are authentic targets of miRNA-661. As expected, immunofluorescence and Western blot approaches revealed a decrease of the Nectin-1 and stard10 protein after SNAI1 induction in MCF7-SNAI1 expressing miRNA-661 (respectively FIG. 3C, and FIG. 3D compare lanes 1 and 2). To further corroborate the regulation of these two candidates by miRNA-661, its action was inhibited in induced MCF7-SNAI1 cells where the miR-661 is up-regulated and the endogenous messengers of the potential target genes were quantified (FIG. 3E). It was reasoned that a specific anti-miRNA-661 LNA inhibitor should protect bonafide mRNA targets of this miRNA from its destabilizing action. As expected, the levels of the mRNAs of all tested genes decreased after SNAI1 induction in MCF-7-SNAI1 cells (FIG. 3E). However, anti-miRNA-661 LNA only inhibited down-regulation of the endogenous messengers of nectin-1 and StarD10 in induced MCF-7-SNAI1 cells whereas the other predicted candidates (NQO2, FLII, RNPEL1, CACNAH1) or the E-cadherin control messengers were not protected by the anti-miR-661 LNA. As expected, StarD10 and nectin-1 levels decreased in induced cells treated with the control LNA (LNA-sc) (FIG. 3E). Immunoblotting analysis of cell extracts obtained under similar experimental conditions, confirmed the stabilization of the Nectin-1 and StarD10 at the protein level after anti-miR-661 LNA treatment (FIG. 3D). To determine if the negative regulation of these two genes is an early event occurring during EMT-induced by SNAI1, the endogenous mRNA levels of the Nectin-1 and StarD10 were monitored in a time course experiment, after SNAI1 induction in MCF-7-SNAI1. Nectin-1 mRNA level decreased between 8 h and 12 hours after SNAI1 expression and StarD10 level between 12 h and 24 h in induced MCF-7-SNA11 cells (FIG. 4B), suggesting an early negative regulation. Because these two genes are repressed during epithelial transition in the MCF-7-SNAI1 cells, it was decided to evaluate if their expressions are inversely correlated with the expression of miRNA-661 which is specifically expressed in invasive breast carcinoma cells (FIG. 2C). Importantly, an inverse correlation was found between the expression of miR-661 and the expression of Nectin-1 and StarD10 in non-invasive and invasive epithelial cell lines (compare FIG. 2C to FIG. 4A). Indeed, Nectin-1 and StarD10 were expressed in carcinoma cell lines exhibiting an epithelial phenotype at various levels, but not in the mesenchymal-like and invasive MDA-231 and MDA-435 cell lines (FIG. 4A) Collectively, these results show that one mechanism of regulation of the translation of nectin-1 and StarD10 messengers is their destabilization mediated by miR-661 in epithelial cells which undergo an EMT transition. In addition, their negative regulation being an event occurring early after the SNAI1-induction, supports the view that their expression is important to maintain an epithelial phenotype and/or to promote the epithelial to mesenchymal transition.

Discussion

Malignant cancer cells are known to reactivate a program leading to EMT which plays a crucial role during embryonic development and which can also be activated in a limited subset of cells in adults, like for example, during wound healing. Using breast cancer cells conditionally expressing SNAI1, novel miRNAs was identified which are implicated in the regulation of EMT-associated events. The studies were focused on miRNAs which were up-regulated after the induction of SNAI1 and which are expected to target messengers of genes participating in repressing EMT and cell invasion. The results suggest that miRNA-661 which was so far not described to be associated with EMT, is an important player in the regulatory network leading to EMT and cancer cell invasion. In support of a role in these processes, expression of miRNA-661 highly correlated with the invasive status of breast cancer cell lines. Concordantly, inhibition of its action by a specific LNA revealed its direct implication in cell motility and invasion. In line, forced expression of the miRNA-661 was not sufficient to trigger invasion or migration, or changes in phenotype of MCF-7 cells

Dissociation of cell to cell junctions is one of the main features observed during EMT and cancer cell evasion. The present results showed that the miRNA-661 regulates the stability of the nectin-1 at the protein level. Nectins are immunoglobulin(Ig)-like cell adhesion (CAMs) comprising a family of four members, including nectin-1. These proteins participate in the formation of adherens and tight junctions and regulate epithelial cell polarization, cytoskeleton organization and cell migration. In contrast to Nectin-1, few data are available for StarD10, the second target of miR-661 which was identified in the MCF-7 SNAI1 model. The StarD10 protein mediates lipid transfer between intracellular membranes, a process, which may contribute to processes such as epithelial cell polarity and signaling (Olayioye et al, 2005). Consistent with previous data (Olayioye et al, 2004), it was found that StarD10 protein is over-expressed in weakly invasive breast cancer cells such as MCF-7 or T47D cells when compared to normal breast epithelial cells. Interestingly, StarD10 expression was strongly repressed in the highly invasive MDA-231 and MDA-435 cells ((Olayioye et al., 2004)). These observations, together with the present findings showing that StarD10 is negatively regulated by the miR-661, indicate that the decrease of this protein may specifically contribute to EMT and cell invasion, while high levels of StarD10 might be required for cell proliferation.

Detection of miRNAs for Diagnosis and Prognosis

Determining miRNA profiles might be indicative of the existence or the severity of a particular pathology. Two techniques can be envisaged to measure miRNA expression in patient samples.

Example of miRNA-661 Quantification

Total RNA are extracted using conventional purification methods. First-strand cDNA synthesis is carried out with 250ng of total RNAs in 7.5 nl of final volume containing 50 nM stem-loop primer, 1× RT buffer, dNTPs, RT and RNase inhibitor (FIG. 1). The mix is incubated in PCR tubes at 16° C. for 30 min, 42° C. for 30 min, 85° C. for 5 min, and then held at 4° C. Real-Time PCR is next performed and the 10 nl PCR reactions included 2 ̂l of RT products, 1.5 uM forward primer and 0.7 uM reverse primer. The reactions are incubated at 95° C. for 10 min, followed by 40 cycles of 95° C. for 30 s, 58° C. for 1 min, and 72° C. for 1 min. All reactions are performed in triplicate. The threshold cycle (TC) is defined as the fractional cycle number at which the fluorescence passes the fixed threshold.

In Situ Hybridization

After discovery of miRNAs, a major limitation for understanding miRNA function was the difficulty in determining spatial expression patterns. However, two similar approaches for detection of mature miRNAs by in situ hybridization (ISH) have been recently described: one ISH method is based on Locked Nucleic Acid (LNA) oligonucleotide probes (DNA probes) (Ason et al., 2006, Nelson et al., 2006, Pillai, 2005, Wienholds et al., 2005), and the second one is based upon RNA oligonucleotide probes (Deo et al., 2006, Thompson et al., 2007). These methods were used for detection of mature miRNAs in embryos, in tissue sections from embryonic and adult mice, as well as from human brains sections. The LNA-based miRNA ISH method ensures a high degree of sequence specificity from the base-pairing properties of digoxigenin (DIG) or fluorescein-labeled LNA probes. The miRNA ISH using RNA probes, labelled with either fluorescein or 33P (5′ end), uses high-stringency wash conditions based on tetramethylammonium chloride (TMAC) in combination with RNase A treatment to remove unhybridized probe and to generate highly sequence specific conditions. Both methods appear to generate similar results based on the comparison of published expression patterns.

Example of In Situ Hybridization

Sections of fresh-frozen tissues are prepared using standard protocols (e.g. fixation in 4% paraformaldehyde (PFA), treatment with proteinase K, re-fixation with 4% PFA). Slides are incubated in hybridization buffer for 1-3 h before the addition of the probe (500 000 cpm of 33P-labeled RNA probe or DIG-labeled LNA probe and 1 ng/ml of fluorescein-labeled RNA probe). Slides are then washed in SSC buffer, dehydrated through a graded series of 50-100% ethanol, air dried, and exposed to X-ray film for several days (exposure times can vary depending upon the relative abundance of each miRNA within tissue areas). For the detection of fluorescein-labeled probes, a supplemental step with incubation of slides with an anti-fluorescein antibody is needed.

In addition, it was shown for the first time that miR-661 as well as its targets contributed to EMT-associated breast carcinoma cell invasion. Importantly, in contrast to Nectin-1, the expression of StarD10 was positively associated with markers of luminal subtypes of breast carcinomas while it negatively correlated with markers of the EMT-related basal-like phenotype.

Nectin-1 and StarD10 participate to SNAI1-elicited MCF7 cell invasion Consistent with the observations made in MCF7-SNAI1 cells, Nectin-1 and StarD10 messengers were found expressed in poorly invasive cells (MCF7 and T47D) which express low levels of miR-661, while an inverse expression pattern was observed in highly invasive breast carcinoma cells (MDA-231 and MDA-435). Next, to investigate the relative contribution of Nectin-1 and StarD10 to EMT-related invasion, these proteins were ectopically expressed in induced MCF7-SNAI1 cells to evaluate whether they may overcome the invasion-promoting effect of miR-661. Cells were transfected with Nectin-1 or StarD10 GFP fusion variants lacking the 3′-untranslated region. The expression of the corresponding mRNAs was quantified by real-time PCR. GFP-fusion proteins exhibited the same sub-cellular localization than the endogenous proteins. Invasion assays showed that forced expression of GFP-Nectin-1 and GFP-StarD10 decreased by 31% and 18% respectively cell invasion in induced MCF7-SNAI1 cells as compared to the GFP control. Altogether, these results show that the repression of Nectin-1 and StarD10 via miR-661 contributes to efficient cell invasion triggered by the SNAI1-mediated EMT program. The dosage of theses proteins Nectin-1 and StarD10 is thus a powerful to diagnose EMT types of cancer.

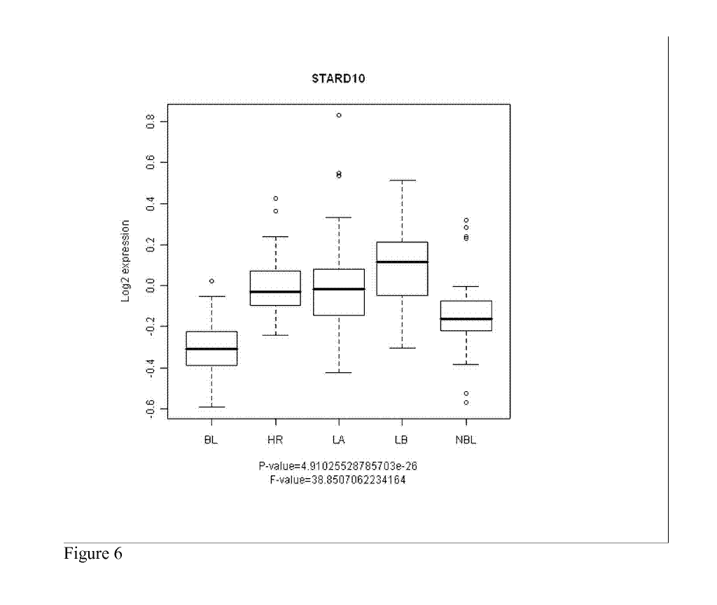

The loss of Nectin-1 and StarD10 in breast carcinoma cells is associated with EMT which is a key step towards metastasis. To evaluate the potential of these proteins in molecular breast tumor subtype classification, a multiclass ANOVA statistical analysis was performed with a previously characterized cohort of 295 breast cancer specimen, classified into cancer subtypes based on gene expression profiles and disease outcome (Fan et al., 2006; van de Vijver et al., 2002). StarD10 showed a strong statistical association with breast cancer subtypes (P-value=4.910E-26), suggesting that StarD10 expression correlated also with the disease outcome in these 295 patients (FIG. 6). In contrast to the cell-cell adhesion molecule Nectin-1 for which no association was determined, the StarD10 was expressed in Luminal A, B (LA and LB) and Her2+ (HR) tumor subtypes (FIG. 6), whereas its expression was low in the basal-like subtype (BL) which has been reported to exhibit molecular characteristics of EMT. The loss of StarD10 may thus be used as a novel molecular marker for the EMT-associated basal-like breast cancer subtype.

The results show that miR-661 is an important player in the regulatory network leading to cancer cell invasion. An interaction between miR-661 and StarD10 or Nectin-1 was experimentally confirmed, and showed that they contribute to cell invasion.

Importantly, the studies leading to the present invention revealed for the first time that the loss of StarD10 expression is a highly relevant marker of human breast cancer for the basal-like subtype. The dosage of StarD10 protein is thus an interesting tool to discriminate between different breast cancer types.

The above-discussed results emphasize the importance of miRNA-triggered down-regulation of cell-cell contact proteins during EMT and cancer cell invasion.

1. miRNA (miRNA661) according to SEQ ID Nr MI0003669 or ENSG00000207574 the sequence of the mature miRNA-661 being 51-ugccugggucucuggccugcgcgu-74 for use as a medicament.

2. A miRNA according to claim 1 for use in the treatment and/or the diagnosis of miRNA associated pathologies including cancer, neuronal disease and infection.

3. A miRNA according to claim 1 or 2 for use in in vitro diagnosis and/or prognosis of cancer, neuronal disease and/or infection.

4. A miRNA according to claim 1 or 2 for use in vitro diagnosis of epithelial to mesenchymal transition of cancer cells.

5. A miRNA according to claim 1 or 2 for use in inhibition of the invasion of breast cancer.

6. A miRNA according to claim 1 or 2 for use as a specific biomarker of invasive breast cancer cells.

7. A miRNA according to claim 1 or 2 for use in regulating the stability of nectin-1 at the protein level.

8. A miRNA according to claim 1 or 2 for use in regulating EMT-associated events.

9. Use of Nectin-1 and/or StarD10 as a molecular marker for breast cancer.

10. Use of StarD10 as a molecular marker of EMT-associated basal-like breast tumor subtype.

11. Use of an anti-SNAI1 antibody, an anti-nectin-1 antibody or anti-StarD10 antibody to detect cancer, in particular breast cancer and more particularly epithelial to mesenchymal transition of cancer.

12. Use of an anti-SNAI1 antibody, an anti-nectin-1 antibody or anti-StarD10 antibody for molecular breast tumor subtype classification

13. A pharmaceutical composition, which comprises a miRNA661 as claimed in any of the claims 1 to 8 and a pharmaceutically acceptable carrier.

14. Method of blocking of miRNA661 by a specific antisense LNA, selected from the group consisting of 5′-A+C+G+CGCAGGCCAGAGACCCA+G+G+C+A-3′.

15. A method of treating miRNA associated pathologies including cancer, neuronal disease and infection a neurodegenerative disease in a patient requiring treatment, which comprises administering an effective amount of a compound as claimed in claim 1.
